(19) United States
(12) Patent Application Publication
(10) Pub. No.: US 2023/0248781 A1
(43) Pub. Date: Aug. 10, 2023

(54) METHOD FOR REGULATING EXPRESSION LEVEL OF MUSASHI1 IN CELLS

(71) Applicant: BEIJING RONGXIANG INSTITUTE OF REGENERATIVE MEDICINE CO., LTD., Beijing (CN)

(72) Inventor: Li LI, Arcadia, CA (US)

(21) Appl. No.: 18/004,828

(22) PCT Filed: Jul. 8, 2021
(86) PCT No.: PCT/CN2021/105234

§ 371 Date: Jan. 9, 2023

(30) Foreign Application Priority Data
Jul. 8, 2020 (CN) 202010649703.2

(51) Int. Cl. A61K 35/644; A61K 31/575; A61K 31/7048; A61K 47/44; A61K 31/366; A61K 31/4748; A61K 31/4375; A61K 31/472; A61K 35/62; A61P 17/02

(52) U.S. Cl. CPC A61K 35/644; A61K 31/575; A61K 31/7048; A61K 47/44; A61K 31/366; A61K 31/4748; A61K 31/4375; A61K 31/472; A61K 35/62; A61P 17/02

(57) ABSTRACT

A method for regulating an expression level of Musashi1 in cells. In particular, the present application relates to a cell-regulating composition capable of regulating the expression or activity, in cells, of any one selected from the followings: K19, α2β1 integrin, and Musashi1. The regulating composition can promote proliferation, growth, and migration of epidermal cells, promote proliferation, growth, and migration of vascular endothelial cells, and promote proliferation, growth, and migration of fibroblasts.

The present application claims the priority of patent application 2020106497032 filed on Jul. 8, 2020.

### FIELD OF THE INVENTION

The present application relates to the field of cell biology. In particular, the present application relates to use of a cell-regulating composition in promoting the expression level of Musashi1 in cells.

### BACKGROUND OF THE INVENTION

The Musashi family is an evolutionarily conserved family of RNA-binding proteins that can be selectively expressed in nervous system stem cells and progenitor cells, and its members include Musashi1 and Musashi2. Musashi1 is the first member of the Musashi family, and was first found in Drosophilidae. Musashi1 and Musashi2 proteins participate in the asymmetric division of stem cells by synergistically activating the Notch signaling pathway via inhibiting the translation process of the target protein Numb mRNAs.

Musashi1, abbreviated as MSI1, is an RNA-binding protein with a molecular weight of 39 KD. It is usually expressed in central nervous system (CNS) stem cells and progenitor cells, and its expression in differentiated cells is down-regulated. Musashi1 is a transcriptional repressor that can directly regulate the expression of the target proteins Numb and P21 (CIP-1). It has been reported in the literature that Musashi1 is expressed in stem cells of a series of tissues such as intestine, breast and hair follicles. In addition, studies have also found that Musashi1 is expressed in lung adenocarcinoma, large cell carcinoma and small cell carcinoma. Recent studies have found that Musashi1 plays a role in regulating apoptosis in ischemic nerve injury. Musashi1 is currently a candidate gene for poorly differentiated cells, and plays an important role in many aspects such as tumor-related signaling pathways, cell proliferation and apoptosis. The expression of Musashi1 gene is high in glioma, esophageal cancer, gastric cancer, colon cancer, breast cancer and other solid tumors. Research on Musashi will provide a new way for in-depth research of tumor genes, and for clinical diagnosis and treatment of tumor-related diseases.

Given the important biological significance of Musashi1, Musashi1-positive cell lines need to be established in the laboratory for study. In view of this, a culture method and reagents for promoting the expression of Musashi1 are needed in the art.

### SUMMARY OF THE INVENTION

The present application provides active ingredients for regulating cells and a use thereof.

According to some embodiments of the present application, provided is a regulating composition capable of regulating the expression or activity, in cells, of any one selected from the following: K19, α2β1 integrin, Musashi1, and a combination thereof.

In some embodiments, the regulating composition is used for one selected from the following or a combination thereof: promoting the growth, proliferation, and migration of epidermal cells, promoting the growth, proliferation, and migration of fibroblasts, and promoting the growth, proliferation, and migration of vascular endothelial cells.

In some embodiments, the regulating composition comprises:

0.5 wt % to 20 wt % of sterol,

0.1 wt % to 2 wt % of baicalin, and

1 wt % to 20 wt % of beeswax.

In some embodiments, the regulating composition further comprises a vegetable oil or animal oil.

In some embodiments, the vegetable oil is selected from the group consisting of corn oil, peanut oil, cotton seed oil, safflower oil, tea tree oil, sesame oil, olive oil and soybean oil.

In some embodiments, the sterol is selected from the group consisting of zoosterol and phytosterol. The sterol used in the present application is obtained from various natural sources. For example, phytosterols can be obtained from processed vegetable oils, such as corn oil, wheat seed oil, soybean extract, rice extract, rice bran oil, rapeseed oil, and sesame oil. There are other sources of sterols, such as marine animals.

In some embodiments, the sterol is selected from the group consisting of natural cholesterol, synthetic cholesterol, and isomers or derivative thereof.

In some embodiments, the sterol is selected from the group consisting of stigmasterol, β-sitosterol, ergosterol, γ-sitosterol, brassicasterol, α-spinachsterol, 24-dehydrocholesterol, poriferasterol, daucosterol, and an isomer thereof or a derivative thereof; most preferably the combination of stigmasterol, β-sitosterol and brassicasterol.

In some embodiments, the amount of the sterol is 1 wt % to 10 wt %, preferably 2 wt % to 6 wt %.

In some embodiments, the regulating composition further comprises 2 wt % to 10 wt % of beeswax, preferably 2% to 10%, most preferably 3% to 6%.

Beeswax is used as an excipient to produce drugs for external use. The composition of beeswax can be classified into 4 categories, namely lipids, free acids, free alcohols and hydrocarbons. Beeswax also contains trace amounts of volatile oils and pigments.

In the present application, beeswax provides a support structure for the sterol in the regulating composition. Beeswax can form a three-dimensional structure comprising an oil in which the sterol is dissolved.

The regulating composition can contain a small amount of water, less than 0.5% of water by weight, preferably less than 0.1%.

In some embodiments, the regulating composition further comprises 0.1 wt % to 30 wt % of propolis, preferably 1% to 20%, most preferably 5% to 10%.

In some embodiments, the amount of the baicalin is 0.2 wt % to 1 wt %, preferably 0.2 wt % to 1 wt %, more preferably 0.5 wt % to 1 wt %. The baicalin can be extracted from Scutellaria baicalensis Georgi (Chinese Dictionary of Traditional Chinese Medicine, Shanghai Science and Technology Press, 1986, pp. 2017-2021). It can be extracted using oil, ethanol or other organic solvents, preferably an oil at 100° C. (more preferably at a temperature between 120° C. and 200° C., most preferably at a temperature between 160° C. and 180° C.).

In some embodiments, the regulating composition further comprises 0.1 wt % to 2 wt % of obaculactone, preferably 0.2 wt % to 1 wt %, more preferably 0.5 wt % to 1 wt %. The obaculactone can be extracted from Phellodendron amurense Rupr (Chinese Dictionary of Traditional Chinese Medicine, Shanghai Science and Technology Press, 1986, pp. 2031-2035). It can be extracted using oil, ethanol or other organic solvents, preferably an oil at 100° C. (more preferably at a temperature between 120° C. and 200° C., most preferably at a temperature between 160° C. and 180° C.).

In some embodiments, the regulating composition further comprises 0.001 wt % to 2 wt % of obaberine, preferably 0.002 wt % to 0.5 wt %, more preferably 0.003 wt % to 0.1 wt %. The obaberine can be extracted from Scutellaria baicalensis Georgi, Phellodendron amurense Rupr and/or Coptis chinensis Franch (Chinese Dictionary of Traditional Chinese Medicine, Shanghai Science and Technology Press, 1986, pp. 2022-2030). It can be extracted using oil, ethanol or other organic solvents, preferably an oil at 100° C. (more preferably at a temperature between 120° C. and 200° C., most preferably at a temperature between 160° C. and 180° C.).

In some embodiments, the regulating composition further comprises 0.001 wt % to 2 wt % of berberine, preferably 0.002 wt % to 0.5 wt %, more preferably 0.003 wt % to 0.1 wt %.

In some embodiments, the regulating composition further comprises 0.001 wt % to 2 wt % of papaverine, preferably 0.002 wt % to 0.5 wt %, more preferably 0.003 wt % to 0.1 wt %.

In some embodiments, the regulating composition further comprises 0.001 wt % to 2 wt % of earthworm, preferably 0.002 wt % to 0.5 wt %, more preferably 0.003 wt % to 0.1 wt %.

In some particular embodiments, provided is a regulating composition comprising or consisting of the following:

2 wt % to 6 wt % of sterol,

0.5 wt % to 1 wt % of baicalin,

3 wt % to 6 wt % of beeswax,

5 wt % to 10 wt % of propolis,

0.5 wt % to 1 wt % of obaculactone,

0.003 wt % to 0.1 wt % of obaberine,

0.003 wt % to 0.1 wt % of berberine,

0.003 wt % to 0.1 wt % of papaverine,

0.003 wt % to 0.1 wt % of earthworm, and

a vegetable oil or animal oil.

According to some embodiments, provided is a method for regulating cells in situ, ex vivo or in vitro, including a step of exposing the cells to the regulating composition of the present application.

According to some embodiments, provided is a method for increasing the expression level of Musashi1 in cells in situ, ex vivo or in vitro, including a step of exposing the cells to the regulating composition of the present application.

In some embodiments, the cells are mammalian cells selected from the group consisting of mechanically injured skin cells, chemically injured skin cells, thermo injured skin cells, and skin cells from patients with diabetes.

In some embodiments, the mammalian cells are selected from the group consisting of epidermal cells, granulation tissue cells, and vascular endothelial cells.

In some embodiments, provided is a method for increasing the expression level of Musashi1 in cells in situ, ex vivo or in vitro, including the following steps:

a) optionally, isolating mammalian cells from mammals;

b) exposing the mammalian cells to the regulating composition of the present application, for at least 10 days, preferably 10 to 60 days.

In some embodiments, the exposure lasts for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days, or more.

In some embodiments, the exposure is carried out at 30 ° C. to 40 ° C., preferably 35 ° C., 36° C., 37° C., 38° C.

In the present application, the conditions for maintaining the cells are not particularly limited, and conventional methods suitable for maintaining epidermal cells, granulation tissue cells, or vascular endothelial cells in the art are also applicable to the method of the present application.

In some embodiments, “regulating” refers to one selected from the following or a combination thereof: promoting the growth, proliferation, and migration of epidermal cells, promoting the growth, proliferation, and migration of fibroblasts, and promoting the growth, proliferation, and migration of vascular endothelial cells.

In some embodiments, the regulating composition increases the expression of K19 in epidermal cells.

In some embodiments, the regulating composition increases the expression of α2β1 integrin in granulation tissues.

In some embodiments, the regulating composition increases the expression of Musashi1 in granulation tissue cells.

In some embodiments, the regulating composition increases the expression of Musashi1 in vascular endothelial cells.

According to some embodiments, also provided is a cell culture medium comprising the composition for regulating the expression level of Musashi1 according to the present application.

According to some embodiments, the culture medium of the present application is suitable as an in vitro cell growth culture medium, and for in vitro reconstruction of tissues and/or organs.

In some embodiments, the cell culture medium optionally also comprises various amino acids, for example 18 natural amino acids, thereby providing nutritional support for cell growth. The amino acids can be chemically synthesized or naturally occurred.

In some embodiments, the cell culture medium optionally also comprises nucleotides or bases, such as adenine, cytidine, guanine, thymine, and uridine.

In some embodiments, the cell culture medium optionally also comprises enzymes or cytokines, thereby supporting cell growth and maintaining the desired balance.

According to some embodiments, provided is the regulating composition according to the present application for use in constructing Musashi1-positive cells. In some embodiments, the cells are selected from the group consisting of epidermal cells, granulation tissue cells, and vascular endothelial cells.

According to some embodiments, provided is the use of the regulating composition according to the present application in constructing K1⁹⁺, α2β1 integrin⁺, and Musashi1⁺ triple-positive cells. In some embodiments, the cells are selected from the group consisting of epidermal cells, granulation tissue cells, and vascular endothelial cells.

### DESCRIPTION OF THE DRAWINGS

FIGS. 1A to 1D: HE staining results (10×, patient sample No. 1).

FIGS. 2A to 2D: HE staining results (40×, patient sample No. 1).

FIG. 3: Expression levels of K19 (patient sample No. 1).

FIGS. 4A to 4C: Expression levels of α2β1 integrin (patient sample No. 1).

FIGS. 5A to 5B: HE staining results (10×, patient sample No. 2).

FIGS. 6A to 6B: HE staining results (40×, patient sample No. 2).

FIGS. 7A to 7B: Expression levels of K19 (patient sample No. 2).

FIGS. 8A to 8B: Expression of levels of α2β1 integrin (patient sample No. 2).

FIGS. 9A to 9D: HE staining results (10×, patient sample No. 3).

FIGS. 10A to 10D: HE staining results (40×, patient sample No. 3).

FIGS. 11A to 11D: Expression levels of K19 (patient sample No. 3).

FIGS. 12A to 12D: Expression levels of α2β1 integrin (patient sample No. 3).

FIGS. 13A to 13C: Immunofluorescence staining results for Musashi1. Blue: DAPI (nucleus), red: the position showing Musashi 1 expression, distributed at the nucleus and cytoplasm (patient sample No. 3).

### DETAILED DESCRIPTION OF THE INVENTION

### Example. Preparation of the Regulating Composition

2 wt % to 6 wt % of sterol, 0.5 wt % to 1 wt % of baicalin, 3 wt % to 6 wt % of beeswax, 5 wt % to 10 wt % of propolis, 0.5 wt % to 1 wt % of obaculactone, 0.003 wt % to 0.1 wt % of obaberine, 0.003 wt % to 0.1 wt % of berberine, 0.003 wt % to 0.1 wt % of papaverine, and 0.003 wt % to 0.1 wt % of earthworm were dissolved in a vegetable oil such as soybean oil, sesame oil and corn oil.

Beeswax was heated until melted at 70-80° C. The melted beeswax was mixed with the aforementioned vegetable oil comprising the active ingredients, and gradually cooled to ambient temperature (i.e., 20-25° C.) to obtain the regulating composition of the present application. As beeswax cools faster than oil, the beeswax formed a small “lattice”-like three-dimensional framework structure (in which oil droplets were encapsulated). The dimension of the nest ranged from 5 μm to 50 μm, for example 10 μm to 30 μm, or 15 μm to 20 μm (the detection method for the “lattice”-like three-dimensional framework structure can be found in CN1827766A).

### Test Examples

1. Sample collection:

Patients with Wagner grade 3 diabetic foot (3 cases) were enrolled at the Burn Department of People's Hospital of Ningxia Hui Autonomous Region. All 3 patients signed informed consent forms for the scientific research.

2. Molecules studied: K19, α2β1 integrin, and Musashi1.

3. Sample pretreatment:

Tissue specimens were collected at the junction of the diseased skin and the ulcer at the lesion site using dermatopathological sampling forceps. Three specimens were collected at a time:

(1) one specimen was fixed with formalin and embedded in wax for morphological studies;

(2) two specimens were preserved in liquid nitrogen for molecular biology studies.

4. Grouping and processing methods:

(1) Control group: patients were treated by the conventional treatment method (cleaning the ulcer, making an incision to expose the wound, and applying 5% sulfadiazine zinc ointment to the wound twice a day). The duration of hospitalization and treatment was 30-60 days.

(2) Experimental group: patients were treated with the regulating composition prepared in the Example (cleaning the ulcer, making an incision to expose the wound, and then applying the regulating composition of the present application to the wound twice a day). The duration of hospitalization and treatment was 30-60 days.

### Test Example 1. HE Staining Results

(1) Patient No. 1:

In the experimental group, skin tissues were collected on Days 5, 15 and 20 after treatment. Epidermis and a small amount of dermis were observed. The structure of each layer of the epidermis was intact. The papillary layer and connective tissues can be observed in the dermis layer. The epidermis showed signs of parakeratosis. The results shown were related to an accelerated rate of renewal and proliferation of the epidermis.

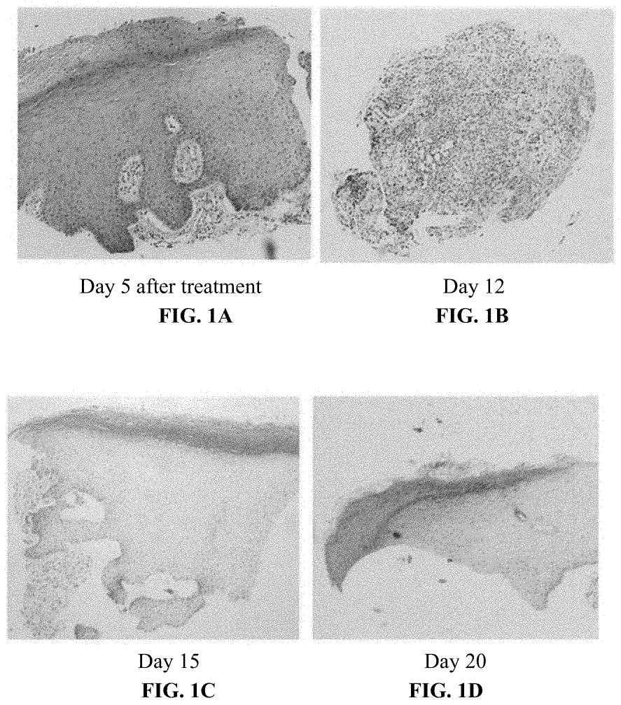

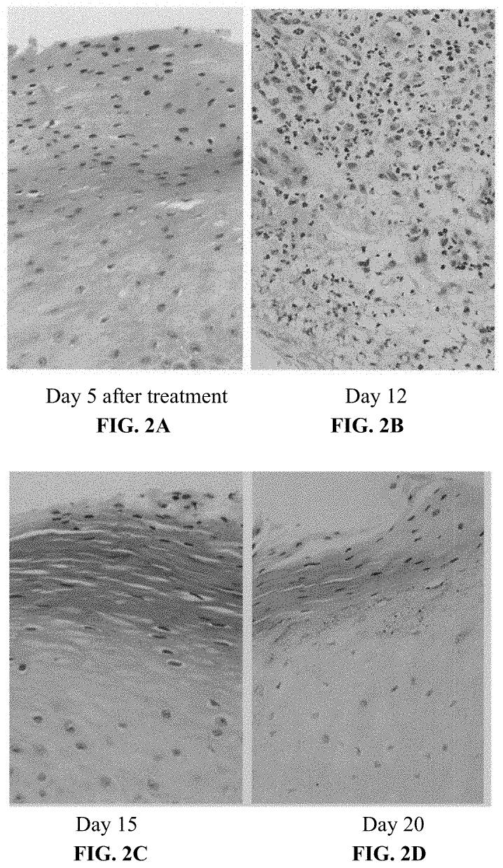

Granulation tissues were collected on Day 12 after treatment. A large number of fibroblasts, inflammatory cells, and capillaries were observed (FIGS. 1A to 1D). The signs of parakeratosis weakened and the epidermal tissue tended to mature on Days 15 and 20, compared with Day 5 after treatment (FIGS. 2A to 2D).

(2) Patient No. 2:

Skin tissues were collected on Days 40 and 55 after treatment. Epidermis and a small amount of dermis were observed. The structure of each layer of the epidermis was intact. The papillary layer and connective tissues were observed in the dermis. The epidermis showed signs of parakeratosis, which was considered to be related to the accelerated rate of renewal and proliferation of the epidermis.

The stratum granulosum was fully differentiated on Days 40 and 55 after treatment, which showed complete repair of the epidermis (FIGS. 5A to 5B, FIGS. 6A to 6B).

(3) Patient No. 3:

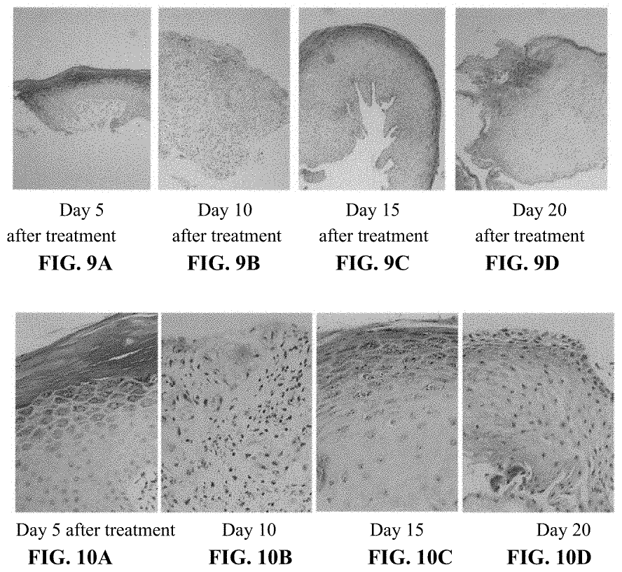

Skin tissues were collected on Days 5, 15 and 20 after treatment. Epidermis and a small amount of dermis were observed. The structure of each layer of the epidermis was intact. The papillary layer and connective tissues were observed in the dermis layer. The epidermis showed signs of parakeratosis, which was considered to be related to the accelerated rate of renewal and proliferation of the epidermis (FIGS. 9A to 9D).

The epidermal structure was intact on Days 5 and 15 after treatment. No stratum corneum was seen in the epidermis on Day 20 after treatment. Granulation tissues were collected on Day 10 after treatment, and abundant fibroblasts and capillaries were observed (FIGS. 10A to 10D).

### Test Example 2. Expression of the Epidermal Cell Marker K19 (Immunofluorescence)

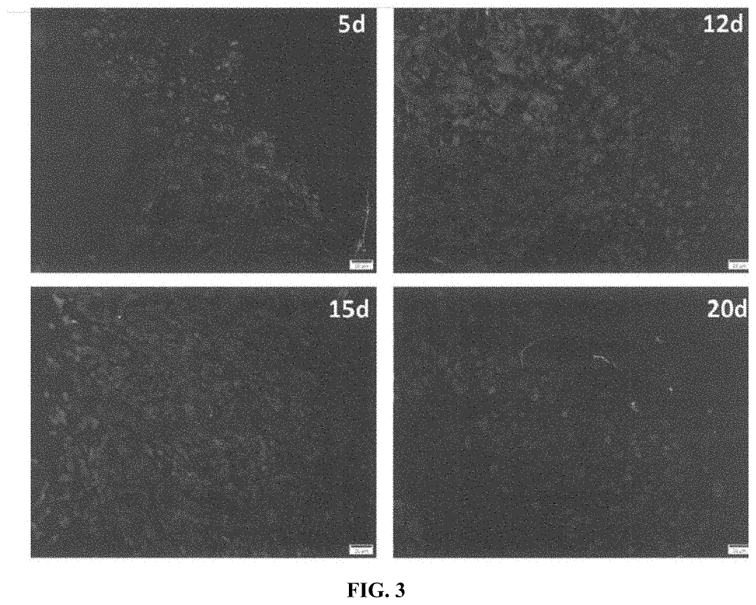

(1) Patient No. 1: The expression level of K19 on Days 12 and 15 after treatment was higher than that of Days 5 and 20 after treatment (FIG. 3).

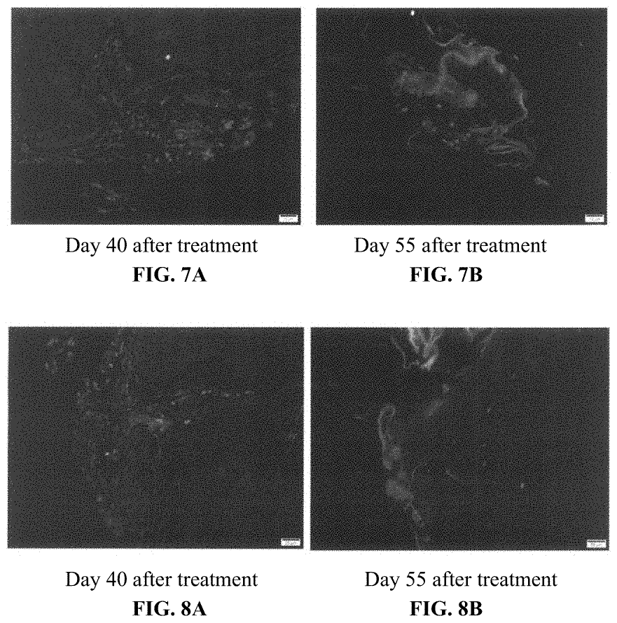

(2) Patient No. 2: The expression of K19 was weak on Days 40 and 55 after treatment. In the papillary layer of the dermis, a low-level expression of K19 was seen in the extracellular matrix, and no expression of K19 was seen in epidermal cells (FIGS. 7A to 7B).

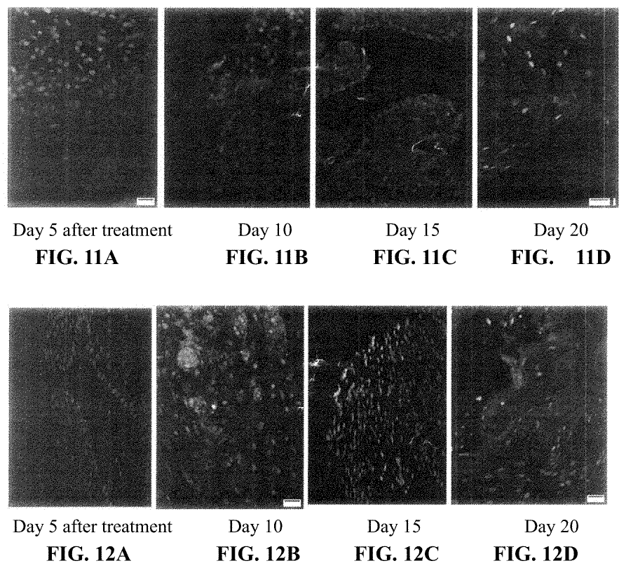

(3) Patient No. 3: The expression of K19 was weak on Days 5, 10 and 20 after treatment, while a strong expression of K19 was observed in the cells of stratum basale of the epidermis on Day 15 after treatment (FIGS. 11A to 11D).

### Test Example 3. Expression of Epidermal Cell Marker α2β1 Integrin (Immunofluorescence)

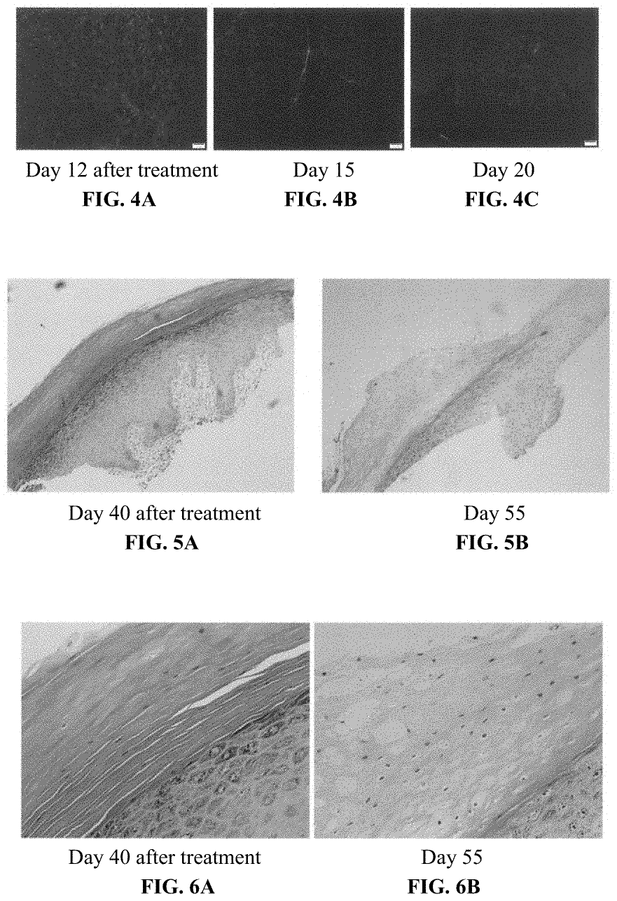

(1) Patient No. 1: Scattered distribution of α2β1 integrin expression was seen in granulation tissues on Day 12 after treatment. There was no positive expression of α2β1 integrin in epidermal cells on Days 15 and 20 after treatment. On Day 20, a weak expression of α2β1 integrin was seen in the extracellular matrix of the dermis (FIGS. 4A to 4C).

(2) Patient No. 2: Almost no expression of α2β1 integrin was seen in the epidermis while a low-level expression was seen in the extracellular matrix of the dermis on Days 40 and 55 after treatment (FIGS. 8A to 8B).

(3) Patient No. 3: α2β1 integrin was almost not expressed in epidermal cells on Days 5, 10, 15 and 20 after treatment, while there was scattered distribution of α2β1 integrin expression in granulation tissues on Day 10 (FIGS. 12A to 12D).

### Test Example 4. Expression of Musashi1 Molecules in Granulation Tissues and Vascular Endothelium (Immunofluorescent Staining)

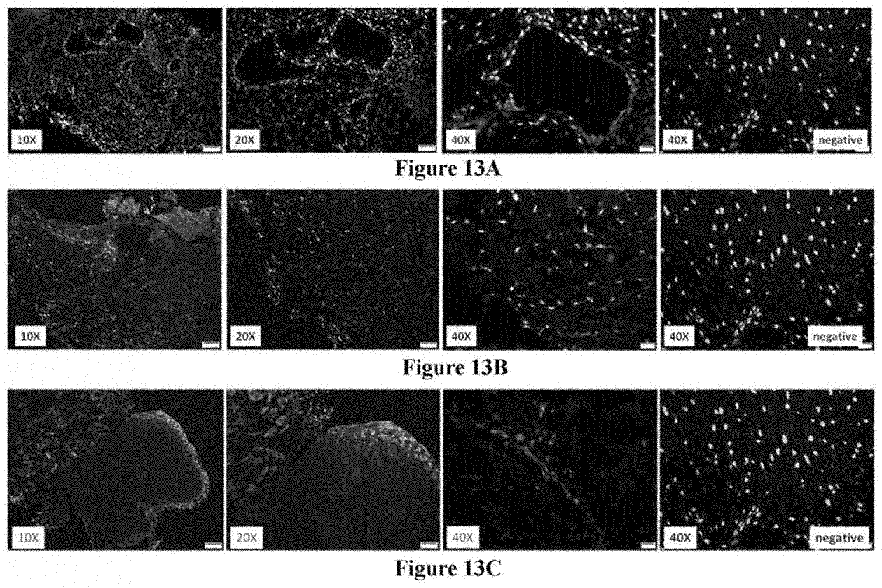

The expression of Musashi1 in granulation tissues was relatively widespread on Day 10 after treatment in the patients (FIG. 13). Musashi1-positive cells were the cells in granulation tissues (no obvious blood vessels were seen), and Musashi1 was widely expressed on Day 15 after treatment in the patients (FIG. 13B). Musashi1-positive cells were the vascular endothelial cells and the cells in granulation tissues, and the expression of Musashi1 was extensive and increased on Day 30 after treatment in the patients (FIG. 13C). In the control samples, the expression of Musashi1 was weak.

In summary, the expression of K19 in epidermal cells at the site of injury was different from that of α2β1 integrin. K19 was expressed mainly in the epidermis, and the expression of K19 on Days 12-15 after treatment was higher than that before administration of the composition of the present application. α2β1 integrin was almost not expressed in the epidermis, but it was expressed in the granulation tissues of the wound, and its expression was significant on Days 10-12 after treatment. The expression of Musashi1 in granulation tissues and vascular endothelial cells increased on Days 10-30 after treatment.

1. A method for regulating the expression level of Musashi1 in cells, including the following steps:
a) optionally, isolating mammalian cells from mammals;
b) exposing the mammalian cells to the regulating composition for at least 10 days, preferably 10 to 60 days;
the mammalian cells are selected from the group consisting of mechanically injured skin cells, chemically injured skin cells, thermo injured skin cells, and skin cells from patients with diabetes;
preferably, the mammalian cells are selected from the group consisting of epidermal cells, granulation tissue cells, and vascular endothelial cells;
the thermo injury is a burn or scald;
the regulating composition comprises (relative to the total weight of the regulating composition):
0.5 wt % to 20 wt % of sterol,
0.1 wt % to 2 wt % of baicalin,
1 wt % to 20 wt % of beeswax, and
a vegetable oil or animal oil;
the vegetable oil is selected from the group consisting of corn oil, peanut oil, cotton seed oil, safflower oil, tea tree oil, sesame oil, olive oil and soybean oil;
the sterol is selected from the group consisting of stigmasterol, β-sitosterol, ergosterol, γ-sitosterol, brassicasterol, α-spinachsterol, 24-dehydrocholesterol, poriferasterol, daucosterol, and an isomer thereof or a derivative thereof; most preferably the combination of stigmasterol, β-sitosterol and brassicasterol;
the regulation refers to increase or promotion;
the method is performed in situ, in vitro or ex vivo.

2. The method according to claim 1, wherein:
the amount of the sterol is 1 wt % to 10 wt %, preferably 2% to 6%;
the amount of the baicalein is 0.2 wt % to 1 wt %, preferably 0.5% to 1%;
the amount of the beeswax is 2 wt % to 10 wt %, preferably 3% to 6%.

3. The method according to claim 1, wherein the regulating composition further comprises:
0.1 wt % to 30 wt % of propolis, preferably 1% to 20%, more preferably 5% to 10%;
0.1 wt % to 2 wt % of obaculactone, preferably 0.2% to 1%, more preferably 0.5% to 1%;
0.001 wt % to 2 wt % of obaberine, preferably 0.002% to 0.5%, more preferably 0.003% to 0.1%;
0.001 wt % to 2 wt % of berberine, preferably 0.002% to 0.5%, more preferably 0.003% to 0.1%;
0.001 wt % to 2 wt % of papaverine, preferably 0.002% to 0.5%, more preferably 0.003% to 0.1%;
0.001 wt % to 2 wt % of earthworm, preferably 0.002% to 0.5%, more preferably 0.003% to 0.1%.

4. The method according to claim 1, wherein the regulating composition can be used to achieve any one selected from the following or a combination thereof:
increasing the expression of K19 in epidermal cells;
increasing the expression of α2β1 integrin in granulation tissue cells;
increasing the expression of Musashi1 in granulation tissue cells;
increasing the expression of Musashi1 in vascular endothelial cells.

5. A composition for regulating the expression level of Musashi1, comprising:
2 wt % to 6 wt % of sterol,
0.5 wt % to 1 wt % of baicalin,
3 wt % to 6 wt % of beeswax,
5 wt % to 10 wt % of propolis,
0.5 wt % to 1 wt % of obaculactone,
0.003 wt % to 0.1 wt % of obaberine,
0.003 wt % to 0.1 wt % of berberine,
0.003 wt % to 0.1 wt % of papaverine,
0.003 wt % to 0.1 wt % of earthworm, and
a vegetable oil or animal oil;
the vegetable oil is selected from the group consisting of corn oil, peanut oil, cotton seed oil, safflower oil, tea tree oil, sesame oil, olive oil and soybean oil;
the sterol is selected from the group consisting of stigmasterol, β-sitosterol, ergosterol, γ-sitosterol, brassicasterol, α-spinachsterol, 24-dehydrocholesterol, poriferasterol, daucosterol, and an isomer thereof or a derivative thereof; most preferably the combination of stigmasterol, β-sitosterol and brassicasterol.

6. A cell culture medium comprising the composition according to claim 5.

7. The cell culture medium according to claim 6, further comprising any one selected from the following: natural amino acid, nucleotide, base, enzyme, cytokine, salt, or a combination thereof.

8. The composition according to claim 5 for use in constructing Musashi1-positive cells, wherein the cells are selected from the group consisting of epidermal cells, granulation tissue cells, and vascular endothelial cells.

9. The composition according to claim 5 for use in constructing K19, α2β1 integrin, and Musashi1 triple-positive cells, wherein the cells are selected from the group consisting of epidermal cells, granulation tissue cells, and vascular endothelial cells.
